SEQUENCE LISTING (2 sequences)

SEQ ID NO: 1 - DNA, 35 bp, Artificial Sequence
  Description of Artificial Sequence Synthetic primer
gatgagttcgtgtccgtacaactggcgtaatcatg

SEQ ID NO: 2 - DNA, 28 bp, Artificial Sequence
  Description of Artificial Sequence Synthetic primer
gacaatctggaatacgccacctgacttg

(19) United States
(12) Patent Application Publication
(10) Pub. No.: US 2012/0270309 A1
(43) Pub. Date: Oct. 25, 2012

(54) THERMAL CYCLER

(75) Inventors: Tateki Arai, Otsu-shi (JP); Osamu Takeda, Otsu-shi (JP); Hiroyuki Izu, Otsu-shi (JP); Hiroyuki Mukai, Otsu-shi (JP)

(73) Assignee: Takara Bio Inc., Otsu-shi (JP)

(21) Appl. No.: 13/389,479

(22) PCT Filed: Aug. 18, 2010
(86) PCT No.: PCT/JP2010/063926

§ 371 Date: Feb. 8, 2012

(30) Foreign Application Priority Data
Aug. 20, 2009 (JP) 2009-190713

(51) Int. Cl. C12M 1/00

(52) U.S. Cl. 435/303.1

(57) ABSTRACT

A thermal cycler for incubating reaction mixture, the thermal cycler including:(1) a heat block for holding and heating reaction containers which contain the reaction mixture, the heat block being a double-layered structure of a lower layer heat block and an upper layer heat block; and (2) a thermal control means for controlling a temperature of the lower layer heat block and a temperature of the upper layer heat block independently and respectively and keeping the temperature of the upper layer heat block higher than the temperature of the lower layer heat block while incubating the reaction mixture. Since the thermal cycler prevents the occurrence of the condensation of water or other components of the reaction mixture in the reaction container and the thermal differences based on the installation positions of the reaction containers, the PCR method and the other enzyme reaction can be performed in a good repeatability.

### FIELD OF THE INVENTION

The present invention relates to a thermal cycler which is useful to react a biological sample, specially to amplify a nucleic acid, in the field of molecular biological study and the like.

### DESCRIPTION OF THE RELATED ART

In the molecular biological study, various chemical reactions such as an enzyme reaction are used for analyzing the sample. It is suitable to achieve reaction with a little sample when the amount of sample is limited.

The nucleic acid amplification reaction is performed in the method which repeatedly synthesizes with a template acid, acids having sequences complementary and/or identical to the template acid. Various methods are developed in different principles, such as PCR method, LCR method, NASBA method, ICAN method, SDA method and LAMP method. Each of them has different features and be used according to the purpose, while the PCR method is used in most situations.

In the PCR method, a reaction mixture including a nucleic acid as a template, a pair of oligonucleotideprimers and heat-resistant DNA polymerase are reacted in the thermal cycle such that “the denaturation of the double-stranded nucleic acid”, “the annealing of the oligonucleotideprimer to the template nucleic aid” and “the synthesis of the complementary acid at the template acid” are sequentially happened. For the purpose the thermal cycler has been developed to temporally and automatically change the temperature of the reaction mixture.

In the PCR method, a slight amount of the reaction mixture (about 10-200 μL) is ordinarily used. In the case of using the capacious reaction container, when thermal differences occur between positions of the reaction container, a condensation of moisture is appeared at the low temperature position, for example on the upper wall of the reaction container noncontact with the reaction mixture, to cause a decrease of amplification efficiency and an unevenness of amplification efficiency between samples. To solve the problems and realize a more precise thermal cycle, Patent document 1 discloses an apparatus including means for covering the upper surface of the reaction container containing the sample with a heated member. Patent document 2 discloses an apparatus heating/cooling the whole of the reaction container by the circulation of the air.

### PRIOR ART DOCUMENTS

### Patent Document

[Patent document 1] Japanese Laid-Open Patent Publication No. 6-233670 A
[Patent document 2] Japanese Laid-Open Patent Publication No. 2000-511435 A

### SUMMARY OF THE INVENTION

### Problems to be Solved by the Invention

The latter apparatus has to include a space for the circulation of air around the reaction container and a complex component having means for the thermal control and circulation of air, and use the highly thermal conductive reaction container (for example the glass container). Meanwhile, the former apparatus has only to include a heat block and a cover, and can be used with a microtube or a microtiter plate which is widely used in the study of biochemistry. However, since a space is made between the heat block and the cover, the temperature of the space lowers so that the condensation of moisture and the like can be caused. Further, the thermal differences of each of the reaction containers are caused between the installation positions thereof, for example between the center position of and the terminal positions of the heat block. Therefore, it has a problem that each of the reactions becomes unevenly between the installation positions of the reaction containers by the PCR method or the enzyme reaction. Further, in a nucleic acid detection method (real time—PCR) which detects a signal during amplification, the signal of the amplification of a target nucleic acid cannot be detected because of covering the upper portion of the reaction container with the cover.

### Means for Solving the Problems

The inventors make the heat block for incubating the reaction mixture which is a double-layered structure including an upper layer heat block and a lower layer heat block, and which sets the temperature of the upper layer heat block higher than the temperature of the lower layer heat block. The inventors find that in this case the condensation of moisture from the reaction mixture can be prevented and the PCR method can be performed in a good repeatability.

The invention relates to a thermal cycler for incubating the reaction mixture. The thermal cycler comprises:
(1) a heat block for holding and heating reaction containers which contain the reaction mixture, the heat block being a double-layered structure of a lower layer heat block and an upper layer heat block; and
(2) a thermal control means for controlling the temperature of the lower layer heat block and the temperature of the upper layer heat block independently and respectively and keeping the temperature of the upper layer heat block higher than the temperature of the lower layer heat block while incubating the reaction mixture.

Accordingly the present invention provides a thermal cycler including a heat block which is a double-layered structure having an upper layer heat block and a lower layer heat block.

The heat block is provided with depressions (wells) which can hold reaction containers containing reaction mixture to be incubated. The wells have shapes corresponding to the reaction containers to hold the microtubes and/or the microtiter plate (96 wells, 386 wells etc.), or capillaries. The reaction container has a capacity of 10-2000 μL per reaction container held by one well. The reaction container can be preferably received by the thermal cycler of the present invention by putting a cap or a seal to prevent water residing in the reaction container from evaporating and going outside.

The heat block is preferably made of highly thermal conductive material. Usually the highly thermal conductive metal heat block (for example aluminum heat block and copper alloy heat block) is used. The upper layer heat block and the lower layer heat block can be made of material same as or different from each other.

The heat block is arranged such that the reaction container passes through the upper layer heat block and contacts with the lower layer heat block. Therefore, the upper layer heat block usually has a thickness of 0.5-1.0 cm, preferably 0.6-0.8 cm. The lower layer heat block has a thickness predetermined to receive the reaction container with the upper layer heat block. Preferably, the heat block is arranged to be a double-layered structure including an upper layer heat block and a lower layer heat block so as to receive the reaction container in the well to nearby the upper edge of the container (for example more than 70% of the height of the container, preferably more than 80%, further preferably more than 85%).

The heat block includes a thermal control means for controlling the temperature of the upper layer heat block and the temperature of the lower layer heat block independently of each other. The thermal control means includes the first regulating means for changing and keeping the temperature of the lower layer heat block, the second regulating means for changing and keeping the temperature of the upper layer heat block, and a control means for independently controlling the both regulating means and temporally changing and keeping the temperature of the both layer heat blocks. The control means includes a computer for memorizing information on a thermal profile and instructing the performance, and a sensor for receiving a real-time temperature of the both layer heat blocks and controlling the temperature. The computer controls the temperature of the both layer heat blocks according to the thermal profile on the basis of the inputted thermal profile information and the real-time thermal data of the both layer heat blocks. In another embodiment of the present invention, the thermal cycler does not have the computer but can control the temperature of the both layer heat blocks independently by connecting the first regulating means, the second regulating means and the sensor with an outside computer through an appropriate interface.

For example the sensor includes a resistance temperature detector using the temperature change of the electric resistance. The sensors are provided on the upper layer heat block and the lower layer heat block respectively and can measure the temperatures of the both layer heat blocks independently. Further, a plurality of the sensors may be provided on the both layer heat blocks.

Each of the first and second regulating means can include a conventional heater or cooler, preferably a Peltier element. The thermal cycler can include a heatsink and cooling fan to promote heat release when cooling the heat block. The first regulating means is preferably provided on the lower surface of the lower layer heat block or provided in the heat block to be flush with the lower surface. These positions do not hinder the thermal controlling of the upper layer heat block. The second regulating means is preferably provided on the upper surface of the upper layer heat block or provided in the heat block to be flush with the upper surface. These positions do not hinder the thermal controlling of the lower layer heat block.

In the thermal cycler including the double-layered structure including an upper layer heat block and a lower layer heat block and the thermal control means, the lower layer heat block holds the lower portion of the reaction container to substantially incubate the reaction mixture. The thermal control means incubates the reaction mixture by the lower layer heat block with using the predetermined thermal profile information. The thermal control means controls the temperature of the upper layer heat block and keeps the temperature thereof higher than the temperature of the lower layer heat block. The upper layer heat block heats the upper portion of the reaction container at the temperature higher than the incubation temperature when the reaction mixture is incubated by the lower layer heat block.

As above stated, in the thermal cycler keeping the temperature of the upper layer heat block higher than the temperature of the lower layer heat block while incubating the reaction mixture, the both layer heat blocks can be contacted with each other if the upper layer heat block does not hinder incubating the reaction mixture by the lower layer heat block according to the thermal profile. However, since the both layer heat blocks are generally made of highly thermal conductive material, when contacting the both layer heat blocks with each other, the heat of the upper layer heat block rapidly conducts to the lower layer heat block so that the temperature of the lower layer heat block cannot be controlled according to the thermal profile. Therefore, the thermal cycler preferably includes a thermal conduction obstructing means for obstructing the thermal conduction between the upper layer heat block and the lower layer heat block. While incubating the reaction mixture, the thermal conduction obstructing means can obstruct or lower the heat conduction from the upper layer heat block to the lower layer heat block, the temperature of the lower layer heat block can be controlled according to the thermal profile, and the reaction mixture can be incubated appropriately.

The thermal conduction obstructing means may be a coating (for example silicon or Teflon(registered trademark)) applied on the lower surface of the upper layer heat block and/or the upper surface of the lower layer heat block for obstructing the thermal conduction. The thermal conduction obstructing means may be a heat insulating material (for example silicon or (heat-proof) polyurethane) disposed between the upper layer heat block and the lower layer heat block for obstructing the thermal conduction. And the thermal conduction obstructing means may be a space formed between the upper layer heat block and the lower layer heat block for obstructing the thermal conduction.

However, if an space (the thickness of the heat insulating material or the highest of the space) between the both layer heat blocks is overlarge by providing the heat insulating material or the space, the temperature of the reaction container positioned between the both layer heat blocks may be lower than the temperature of the reaction mixture and may be reached at the temperature for condensing the component of the reaction mixture while incubating the reaction mixture. Therefore, the space must be provided between the both layer heat blocks to keep the temperature of the reaction container positioned between the both layer heat blocks being higher than the temperature of the condensation (dew-point temperature) of the reaction mixture or the temperature of the reaction mixture while incubating the reaction mixture. Accordingly, the space between the both layer heat blocks may be a minimum required space, for example the length of the space is preferably lower than 15% of the height of the reaction container, to incubate the reaction mixture by the lower layer heat block according to the thermal profile and prevent the condensation of the reaction mixture between the both layer heat blocks.

The temperature of the upper layer heat block may be predetermined to be kept higher than the temperature of the lower layer heat block to incubate the reaction mixture by the lower layer heat block according to the thermal profile. The temperature of the upper layer heat block does not have to be changed sequentially according to the thermal profile of the lower layer heat block. The temperature of the upper layer heat block is predetermined to be higher than the temperature of the lower layer heat block by 3° C., preferably be higher than the temperature thereof by 5° C. For example when the temperature of the lower layer heat block is 40-100° C., the temperature of the upper layer heat block is kept to be 60-120° C. When the temperature of the lower layer heat block is lower than 40° C., the temperature of the upper layer heat block is kept to be 45-60° C. The temperature of the upper layer heat block is generally lower than 115-120° C.

While the heat block incubates the reaction mixture, even if the temperature of the incubation increases to a high temperature (for example more than 70° C.), the temperature of the upper portion of the reaction container is kept to be higher than the temperature of the reaction mixture in the reaction container. As a result, water or other components of the reaction mixture does not condense on the upper portion of the reaction container. Therefore, the concentration change of the components of the reaction mixture is prevented, so that the reaction can be performed in a good repeatability.

In the thermal cycler, since the lower layer heat block is covered with the upper layer heat block thermally controlled, the temperature drop of the lower layer heat block by the ambient temperature reduces, so that the temperature of the lower layer heat block totally becomes uniform. As a result, no thermal differences can be brought between the reaction containers based on the installation positions thereof, for example the center and peripheral position of the heat block, resulting in no differences of the reaction efficiency of the reaction containers.

The thermal cycler does not have to include the cover having the regulating means which covers and heats the upper surface of the reaction container. However, the thermal cycler preferably has the cover for preventing the cap of the reaction container from falling and the seal thereof from removing. The cover preferably includes the regulating means and the control means for keeping the temperature thereof same as the temperature of the upper layer heat block to help and enhance the function of the upper layer heat block. Preferably, the cover presses the reaction containers held in the wells downwardly to enhance the contact and the thermal conduction between the lower portion of the reaction container and the lower layer heat block. If the thermal cycler includes a detecting means (for example spectrophotofluorometer) for optically detecting the signal of the reaction mixture (for example fluorescence), for example the cover can be made of light permeable material or has an opening for passing the signal.

The thermal cycler can be arranged to accommodate every the above elements in a housing. The thermal cycler includes, in addition to the heat block and the thermal control means, preferably a computer, an interface for connecting with an outside computer, an input means (for example keyboard) for inputting the thermal profile, and a display (for example liquid-crystal display) for showing the thermal profile and the situation of the performance thereof. The thermal cycler preferably includes an optical device (for example fiberglass, CCD camera, lens, filter and the like) for monitoring the signals in the reaction mixture, for example fluorescence.

Since the thermal cycler prevents the occurrence of the dew condensation on the upper portion of the reaction container, specially on the inside of the cap or the seal, the signal of the reaction mixture can easily be detected without being interrupted by the dew condensation. Therefore, the device of the present invention is advantageous in case of the thermal cycler including the detecting means for optically and sequentially detecting the progress of the reaction in the reaction container, specially for detecting it from above the reaction container.

### Effect of the invention

The thermal cycler can prevent the occurrence of the condensation of water or other components of the reaction mixture in the reaction container. Since the thermal differences based on the installation positions of the reaction containers can be prevented, the PCR method and the other enzyme reaction can be performed in a good repeatability.

### BRIEF DESCRIPTION OF THE DRAWINGS

FIG. 1 is a perspective view illustrating the embodiment of the thermal cycler of the present invention.

FIG. 2A is a plan view illustrating the upper layer heat block of the thermal cycler.

FIG. 2B is a front view illustrating the upper layer heat block of the thermal cycler.

FIG. 3 is a front view illustrating the lower layer heat block of the thermal cycler.

FIG. 4 is an explanatory view illustrating the upper layer heat block and the lower layer heat block.

FIG. 5 is a sectional view illustrating the heat block accommodating and holding the reaction container.

FIG. 6 is an enlarged view illustrating the heat block which has a space between the upper layer heat block and the lower layer heat block.

FIG. 7A is an enlarged view illustrating the upper layer heat block and the lower layer heat block which contact with each other.

FIG. 7B is an enlarged view illustrating the heat block which has a heat obstructing material between the upper layer heat block and the lower layer heat block.

FIG. 8A is an enlarged view illustrating another embodiment of the heat block.

FIG. 8B is an enlarged view illustrating another embodiment of the heat block.

FIG. 8C is an enlarged view illustrating another embodiment of the heat block.

FIGS. 9I-9IV are explanatory views illustrating various heat blocks used in experiments for confirming reactivity.

FIG. 10 is a schematic view illustrating reaction position in a 96 well plate used in the experiments for confirming reactivity.

FIG. 11 is an electrophoresis photograph after PCR reaction with the various heat blocks used in the experiments for confirming reactivity.

### DETAILED DESCRIPTION OF THE PREFERRED EMBODIMENTS

Hereinafter, a thermal cycler of the present invention will be described with reference to the figures. It will be apparent to those skilled person in the art that modification may be made without departing from the spirit and the scope of the invention.

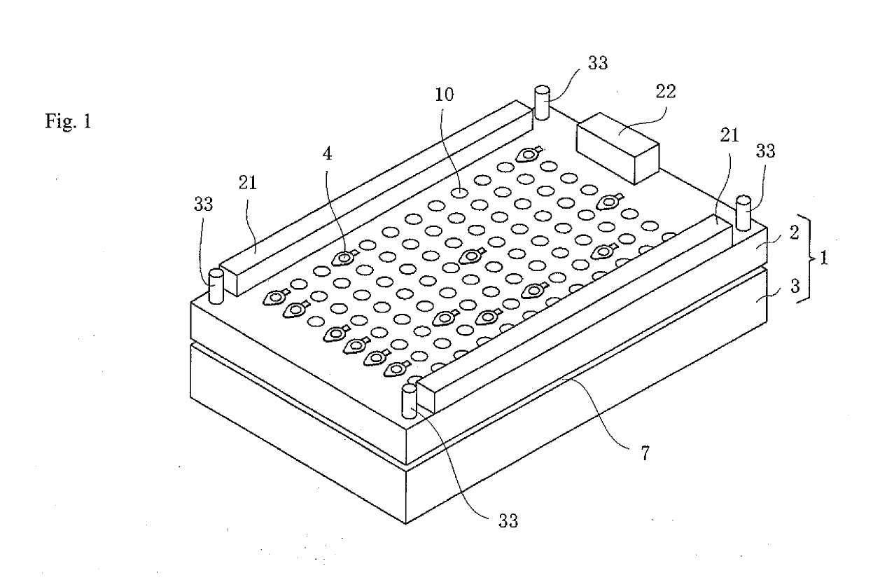

As shown in FIG. 1, the thermal cycler includes a heat block 1 for heating reaction containers 4. The heat block 1 has a plurality of wells 10 for accommodating and holding the reaction containers 4. In the embodiment, the heat block 1 has 96 wells (for 8×12 wells plate). The heat block 1 includes an upper layer heat block 2 and a lower layer heat block 3.

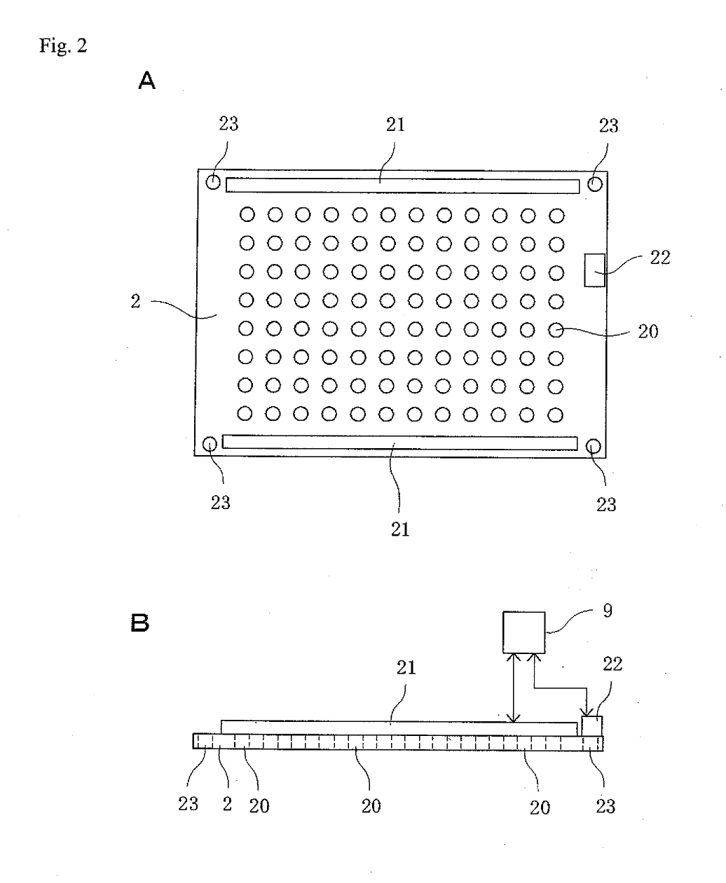

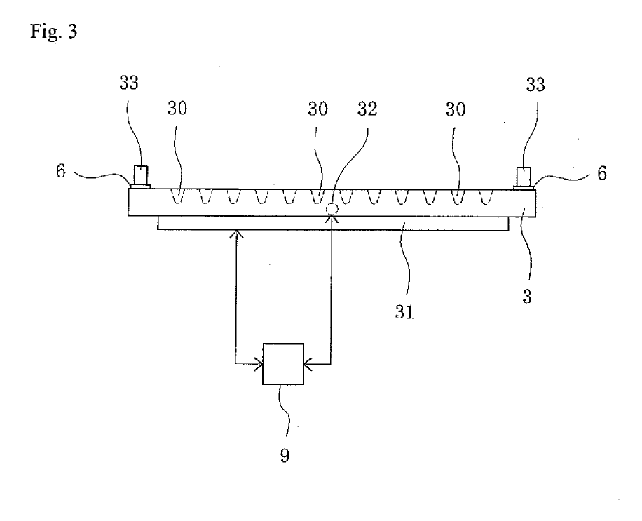

As shown in FIG. 2, the upper layer heat block 2 has through holes 20 which are formed as parts of the wells 10. As shown in FIG. 2B, the through holes 20 extend through the upper layer heat block 2 and have shapes corresponding to the shapes of the peripheries of the upper parts of the reaction containers 4, so that the reaction containers 4 are inserted in the through holes 20 to penetrate therethrough.

The upper layer heat block 2 includes heaters 21 for changing and keeping the temperature thereof, and a thermal sensor 22 for detecting the temperature of the upper layer heat block 2. The heaters 21 and the thermal sensor 22 are disposed in appropriate position to control the temperature of the upper layer heat block 2. In the embodiment, the heaters 21 are disposed on the upper surface of and the widthwise opposite ends of the upper layer heat block 2. The thermal sensor 22 is disposed on the upper surface of and one of the lengthwise opposed ends of the upper layer heat block 2. The heaters 21 and/or the thermal sensor 22 may be disposed on the side surface of the upper layer heat block 2. The upper layer heat block 2 may include accommodating recesses (not shown) for accommodating the heaters 21 and/or the thermal sensor 22 to install them in the upper layer heat block 2.

The heaters 21 and the thermal sensor 22 are connected with a control part 9. The temperature of the upper layer heat block 2 can be controlled at predetermined temperature and sequentially changed by the control part 9.

The upper layer heat block 2 has positioning holes 23 on four corners the upper layer heat block 2. The positioning holes 23 extend through the upper layer heat block 2 to engage and position the lower layer heat block 3.

The lower layer heat block 3 includes recesses 30 which are formed as parts of the wells 10. The recesses 30 are formed into shapes corresponding to the lower parts of the reaction containers 4.

The lower layer heat block 3 has a Peltier element 31 for changing and keeping the temperature of the lower layer heat block 3, and a thermal sensor 32 for detecting the temperature of the lower layer heat block 3. The Peltier element 31 is disposed on the lower surface of the lower layer heat block 3. The thermal sensor 32 is implanted in the center of the lower layer heat block 3.

The Peltier element 31 and the thermal sensor 32 are connected with the control part 9. The control part 9 controls and sequentially changes the temperature of the lower layer heat block 3. The thermal cycler can control the temperature of the upper layer heat block 2 and the lower layer heat block 3 independently and respectively.

The lower layer heat block 3 has positioning shafts 33 for positioning the upper layer heat block 2. The positioning shafts 33 are disposed on the four corners of the upper surface of the lower layer heat block 3. Spacers 6 are disposed on the lower end of each of the shafts 33. A space is provided between the upper layer heat block 2 and the lower layer heat block 3 by the spacers 6 when the upper layer heat block 2 is engaged with the lower layer heat block 3.

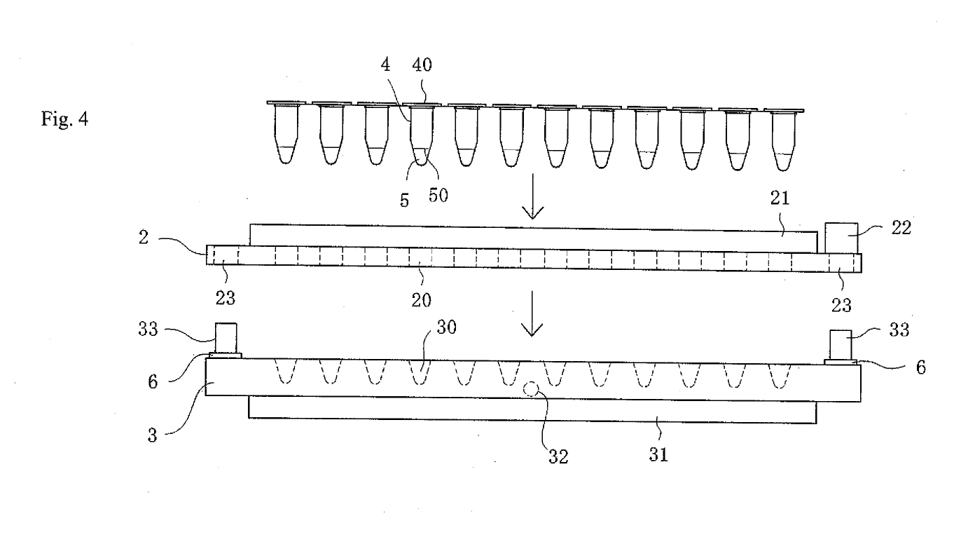

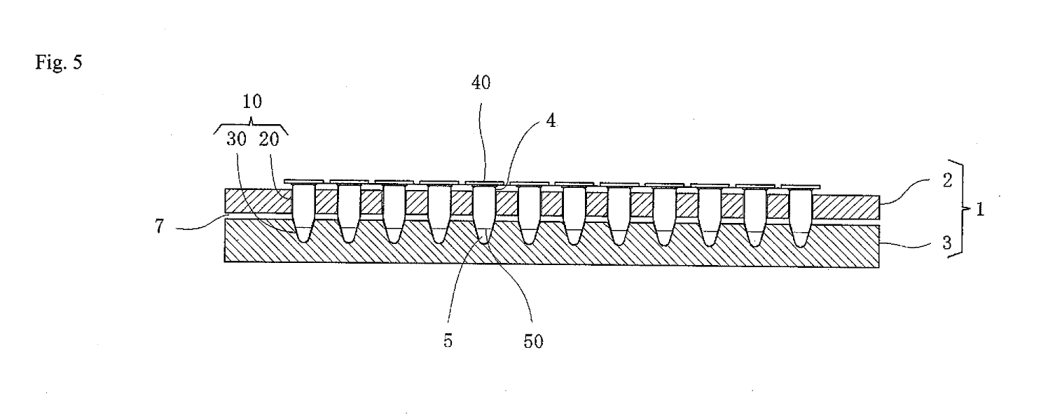

As shown in FIG. 4, the upper layer heat block 2 is disposed on the lower layer heat block 3 such that each of the positioning shafts 33 is fit into each of the positioning holes 23, so that the upper layer heat block 2 is engaged with the lower layer heat block 3. As a result, the upper layer heat block 2 is positioned on the lower layer heat block 3. Therefore, as shown in FIG. 5, each of the through holes 20 of the upper layer heat block 2 is disposed directly above each of the recesses 30 of the lower layer heat block 3 on the same vertical line, so that the wells 10 for accommodating the reaction containers 4 are formed with the through holes 20 and the recesses 30. Since the spacers 6 are interposed between the upper layer heat block 2 and the lower layer heat block 3, the space 7 is provided between the upper layer heat block 2 and the lower layer heat block 3. As a result, as shown in FIG. 1, the heat block 1 is formed with the upper layer heat block 2 and the lower layer heat block 3, the reaction containers 4 containing the reaction mixture 5 are accommodated in the wells 10.

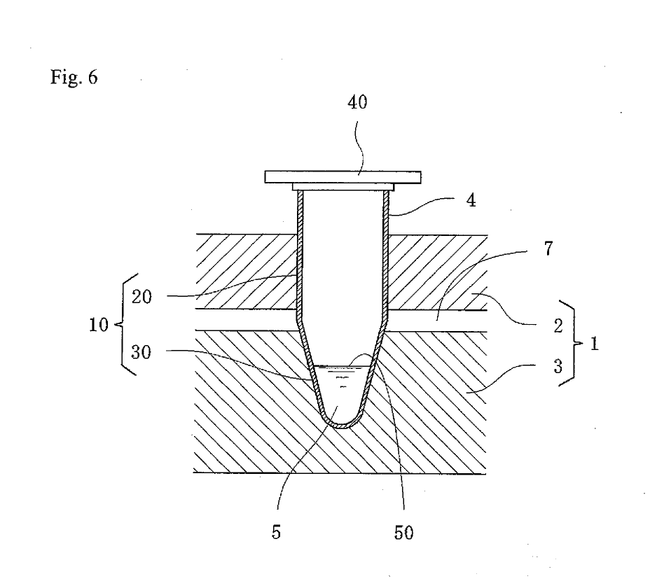

As shown in FIG. 6, the reaction containers 4 containing the reaction mixture 5 with caps 40 are accommodated and held in the wells 10 of the heat block 1 so that the lower parts of the reaction containers 4 are fit in and contacted with the recesses 30 of the lower layer heat block 3. And the peripheries of the upper parts of the reaction containers 4 are contacted with the through holes 20 of the upper layer heat block 2. The caps 40 of the reaction containers 4 are not inserted in the through holes 20 but disposed above the upper layer heat block 2.

The reaction mixture 5 contained in the reaction containers 4 is disposed in the lower layer heat block 3 for incubating. The liquid surface 50 of the reaction mixture 5 is disposed at the same level as, or preferably as shown in FIG. 6 at the lower level than the upper surface of the lower layer heat block 3.

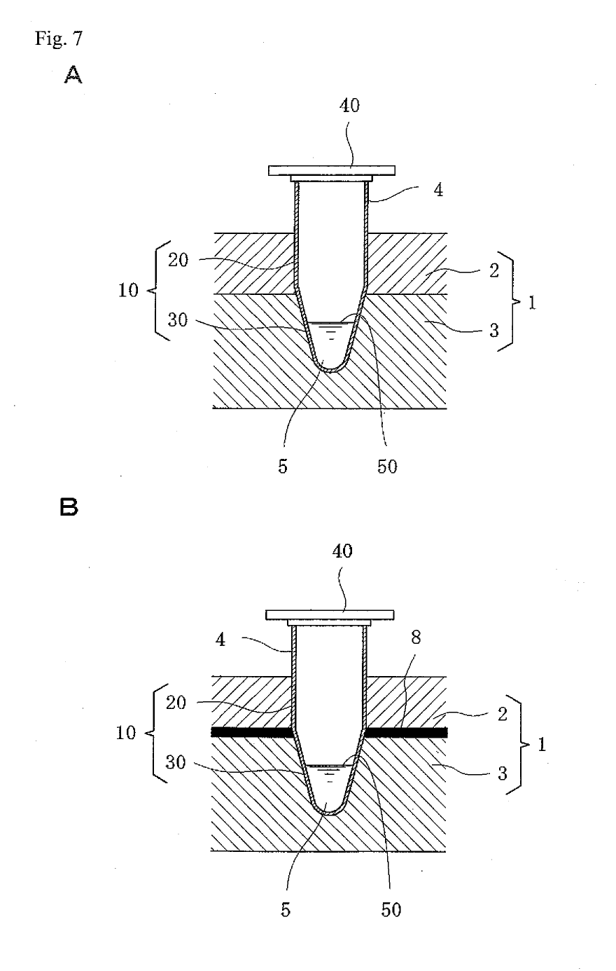

The space 7 disposed between the upper layer heat block 2 and the lower layer heat block 3 has a height capable of obstructing the thermal conduction between the upper layer heat block 2 and the lower layer heat block 3, controlling the temperature according to the thermal profile of the lower layer heat block 3 and preventing the condensation in the reaction containers 4 between the upper layer heat block 2 and the lower layer heat block 3 while incubating. In the case of applying a coating such as Teflon coating on the lower surface of the upper layer heat block 2 and/or the upper surface of the lower layer heat block 3 for obstructing the thermal conduction, the space 7 has not to be provided between the upper layer heat block 2 and the lower layer heat block 3, but as shown in FIG. 7A, the upper layer heat block 2 and the lower layer heat block 3 may be contacted with each other. If the upper layer heat block 2 does not prevent the reaction mixture 5 from being incubated according to the thermal profile by the lower layer heat block 3, the upper layer heat block 2 and the lower layer heat block 3 may be contacted with each other without applying the coating. As shown in FIG. 7B, a heat obstructing material 8 may be provided between the upper layer heat block 2 and the lower layer heat block 3 to obstruct the thermal conduction.

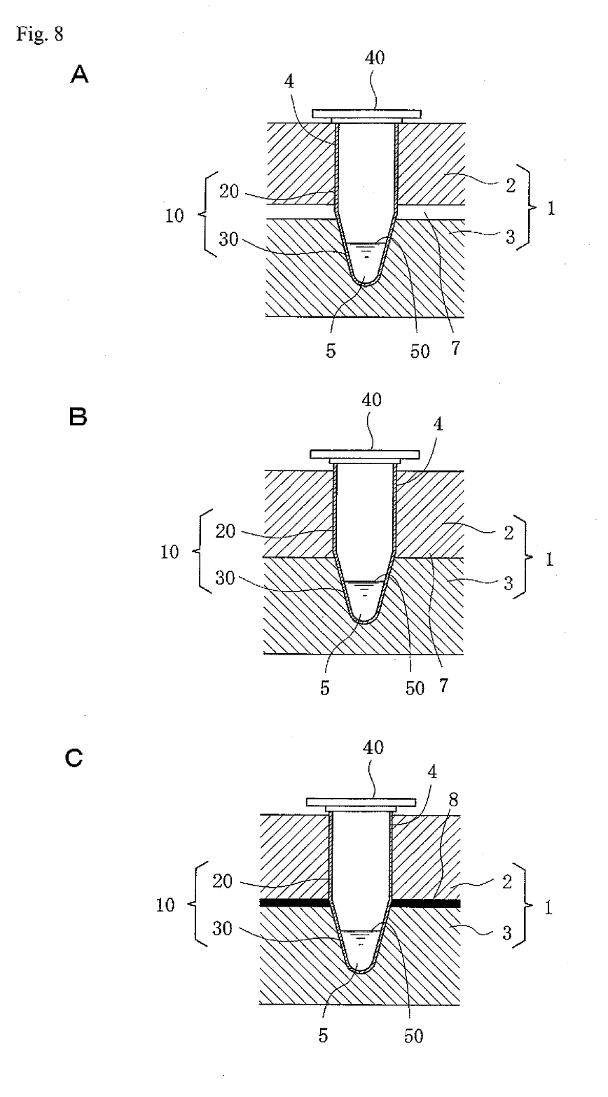

As shown in FIGS. 8A, 8B and 8C, the reaction containers 4 may be accommodated in the heat block 1 to have the upper end thereof positioned near the top of the heat block 1.

The thermal cycler incubates the reaction mixture 5 contained in the reaction containers 4 with the reaction containers 4 held by the heat block 1. The thermal cycler controls the temperature of the lower layer heat block 3 according to the thermal profile inputted in the control part 9, heats the entire lower parts of the reaction containers 4 by the lower layer heat block 3 and incubates the reaction mixture 5. While the lower layer heat block 3 incubates the reaction mixture 5, the thermal cycler controls the temperature of the upper layer heat block 2 and heats the periphery of the upper parts of the reaction containers 4 by keeping the temperature of the upper layer heat block 2 higher than the temperature of the lower layer heat block 3 to prevent the occurrence of the condensation of the reaction mixture 5 in the reaction containers 4.

In the case of amplifying a nucleic acid in PCR method by using the thermal cycler, the temperature of the lower layer heat block 3 is predetermined at 95° C. for 30 seconds in the thermal denature phase, at 55° C. for 30 seconds in the annealing phase and at 72° C. for 1 minute in the extention reaction phase. In this case, the temperature of the upper layer heat block 2 is predetermined to be kept a constant temperature, for example at 105° C., higher than the upper limit temperature (95° C.) of the lower layer heat block 3.

The temperature of the upper layer heat block 2 may be sequentially changed according to the change of the temperature of the lower layer heat block 3 by providing the Peltier element in stead of the heater 21 on the upper layer heat block 2. For example, if the temperature of the lower layer heat block 3 is predetermined at the temperature as above stated, the temperature of the upper layer heat block 2 is predetermined at 105° C. for 30 seconds in the thermal denature phase, at 65° C. for 30 seconds in the annealing phase and at 82° C. for 1 minute in the extention reaction phase. The temperature of the upper layer heat block 2 may be kept higher than the temperature of the lower layer heat block 3 at all time while being changed according the change of the temperature of the lower layer heat block 3.

### [Experiments for Confirming Reactivity]

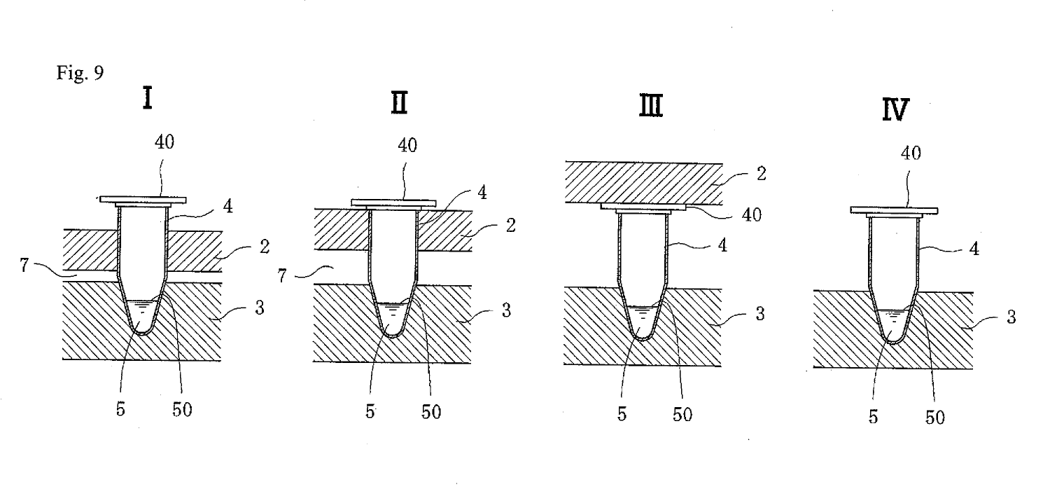

In order to confirm the reactivity of the thermal cycler of the present invention, some thermal cyclers were used with the upper layer heat block 2 as shown in FIG. 9I-9IV. FIG. 91 shows the thermal cycler I of the above stated embodiment shown in FIG. 6. FIG. 9II shows the thermal cycler II which has the upper layer heat block 2 disposed on the uppermost part of the reaction containers 4, and the space 7 disposed between the upper layer heat block 2 and the lower layer heat block 3 and having height corresponding to 30% height of reaction containers 4. FIG. 9III shows the thermal cycler III which has the upper layer heat block 2 disposed above the cap 40 of the reaction containers 4 not to hold the reaction containers 4. FIG. 9IV shows the thermal cycler IV which does not have the upper layer heat block 2.

Amplification efficiency of each of the thermal cyclers was confirmed by PCR amplification reaction of amplification chain length 8 kbp using a lambda DNA (produced by Takara Bio Inc.) as below stated as the template.

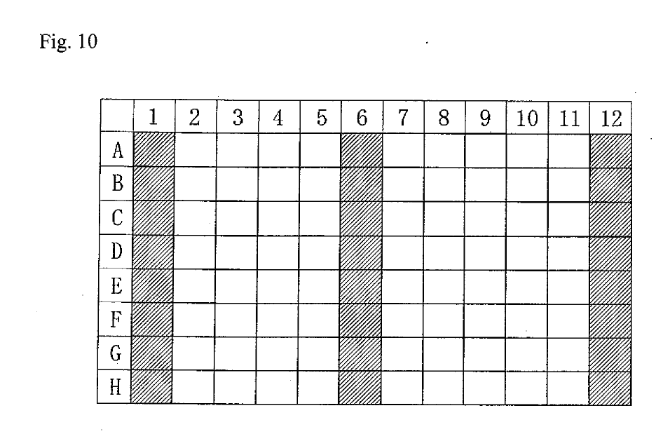

In the reaction, TaKaRa Taq Hot Start Version (produced by Takara Bio Inc.) was used, and the ½ amount (the total reaction mixture amount 25 μL) of general PCR reaction mixture amount described in the manual was used. A lambda DNA of 0.5 μL (2.5 ng/μL) was used as the template, a primer F (SEQ ID NO:1) and a primer R (SEQ ID NO:2) of each of 0.5 μL (10 pmol/μl) were used. The coordinated reaction mixture 5 as above stated was distributed to each of 0.2 mL reaction tubes (0.2 mL 8-strip tube, individual Flat Caps produced by Takara Bio Inc.) by 25 μL. The reaction tubes 4 with the reaction mixture 5 were set on each of the thermal cyclers, and in the reaction the lower layer heat block 3 was heated at 94° C. for 1 minute and then heated in 30 cycles which repeated at 94° C. for 30 seconds—at 65° C. for 10 minutes. In the reaction the upper layer heat block 2 was heated at 107° C. FIG. 10 is a schematic view illustrating a 96 well plate used in the experiments. In FIG. 10 hatching areas show as reaction positions.

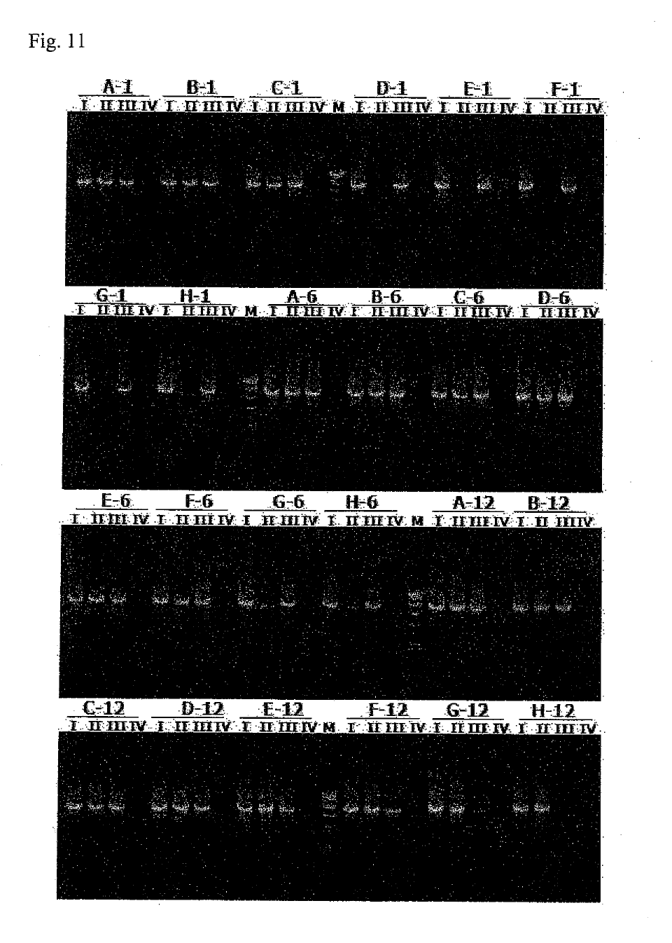

After the reaction, the each reaction mixture from A to H rows of 1st column, 6th column and 12th column in FIG. 10 was extracted, and then the reaction mixture of 3 μL is applied to 1% agarose gel (Agarose L03 “TAKARA” produced by Takara Bio Inc.)/TAE buffer respectively. A λ-Hind III digest (produced by Takara Bio Inc.) was used as the marker, and an electrophoresis was run by a Mupid-2plus (produced by ADVANCE Co., LTD). FIG. 11 shows the result. FIG. 11 is an electrophoresis photograph illustrating the reactivity of the PCR at each certain position of the 96 well plate of each thermal cycler. In FIG. 11 the result after the reaction, for example at A row and 1st column by the thermal cyclers I-IV, is shown in the electrophoresis photograph below “A-1”. And “M” shows the electrophoresis photograph of the marker.

As shown in FIG. 11, it will be apparent that an amplification was little or not recognized at the positions of D to H rows of 1st column and G and H rows of 6th column by the thermal cycler II, at the positions of F to H rows of 12th column by the thermal cycler III and at the positions of every row and column by the thermal cycler IV. It can be recognized that the results happened because the condensation couldn't be prevented in the reaction containers 4 or the thermal differences occurred in the lower layer heat block 3. Meanwhile, the thermal cycler I of the present invention could get good results in the amplification on every position of the 96 well plate. The results may come from preventing the condensation of the reaction mixture in the reaction container by the upper layer heat block 2 and obstructing the thermal differences of the lower layer heat block 3. In the thermal cycler I of the present invention, the upper layer heat block 2 can prevent the concentrations of the reaction mixture 5 from changing and prevent the amplification efficiency of the nucleic acid from lowering by changing the concentrations of the reaction mixture 5. Further, the upper layer heat block 2 can prevent the thermal differences from occurring based on the differences of the reaction positions of the heat block 1 and the amplification efficiency of the nucleic acid from lowering by the differences of the reaction positions. As above stated, it will be apparent that the thermal cycler of the present invention can run the PCR in the 96 well plate in a good stability and repeatability.

The thermal cycler of the present invention can be applied not only to the amplification of the nucleic acid but also to an enzyme reaction such as a reverse transcription reaction.

### INDUSTRIAL APPLICABILITY

The present invention can provide the thermal cycler which prevents the condensation of water and other components of the reaction mixture in the reaction containers and the thermal differences based on the installation positions of the reaction containers. The thermal cycler of the present invention is so useful to react a biological sample, specially to amplify the nucleic acid, in the field of molecular biological study and the like.

### DESCRIPTION OF THE REFERENCE CHARACTERS

1 heat block

2 upper layer heat block

20 through hole

21 heater

22 thermal sensor

23 positioning hole

3 lower layer heat block

30 recess

31 Peltier element

32 thermal sensor

33 positioning shaft

4 reaction container

40 cap

5 reaction mixture

50 liquid surface

6 spacer

7 space

8 heat obstructing material

9 control part

### SEQUENCE LISTING FREE TEXT

SEQ ID NO:1; Primer F to amplify lambda DNA.
SEQ ID NO:2; Primer R to amplify lambda DNA.

1. A thermal cycler for incubating reaction mixture, the thermal cycler comprising:
a heat block for holding and heating reaction containers which contain the reaction mixture, the heat block being a double-layered structure of a lower layer heat block and an upper layer heat block;
the lower layer heat block having recesses for fitting in and holding lower parts of the reaction containers, to heat the lower parts of the reaction containers contacted with the recesses,
the upper layer heat block having through holes for inserting and holding upper parts of the reaction containers, to heat peripheries of the upper parts of the containers contacted with the through holes, and positioned between upper ends of the reaction containers and the lower layer heat block while inserting the reaction containers; and
a thermal control means for controlling a temperature of the lower layer heat block and a temperature of the upper layer heat block independently and respectively and keeping the temperature of the upper layer heat block higher than the temperature of the lower layer heat block while incubating the reaction mixture.

2. The thermal cycler according to claim 1,
wherein the reaction mixture includes an nucleic acid, the lower layer heat block incubating the reaction mixture, the upper layer heat block preventing the amplification efficiency of the nucleic acid from lowering.

3. The thermal cycler according to claim 1, further comprising
thermal conduction obstructing means for obstructing thermal conduction between the lower layer heat block and the upper layer heat block.

4. The thermal cycler according to claim 3, wherein the thermal conduction obstructing means comprises a space formed between the lower layer heat block and the upper layer heat block.

5. The thermal cycler according to claim 1,
wherein the thermal control means includes the first regulating means for changing and keeping the temperature of the lower layer heat block, and the second regulating means for changing and keeping the temperature of the upper layer heat block,
the first regulating means being disposed on a lower side of the lower layer heat block,
the second regulating means being disposed on an upper side of the upper layer heat block.

6. The thermal cycler according to claim 1,
wherein the thermal control means keeps the temperature of the upper layer heat block being 3° C. higher than the temperature of the lower layer heat block.

7. The thermal cycler according to claim 2, further comprising
thermal conduction obstructing means for obstructing thermal conduction between the lower layer heat block and the upper layer heat block.
